(19) United States
(12) Patent Application Publication
(10) Pub. No.: US 2025/0367389 A1
(43) Pub. Date: Dec. 4, 2025

(54) METHOD FOR CONTROLLING ELECTRICAL TREATMENT DEVICE WITH INJECTION FUNCTION AND ELECTRICAL TREATMENT DEVICE

(71) Applicant: SHENZHEN PENINSULA MEDICAL GROUP, Shenzhen (CN)

(72) Inventors: Yanan LI, Shenzhen (CN); Li LIAO, Shenzhen (CN); Xiaobing LEI, Shenzhen (CN); Yi DING, Shenzhen (CN)

(73) Assignee: SHENZHEN PENINSULA MEDICAL GROUP, Shenzhen (CN)

(21) Appl. No.: 19/303,447

(22) Filed: Aug. 19, 2025

Related U.S. Application Data
(63) Continuation of application No. PCT/CN2024/122903, filed on Sep. 30, 2024.

(30) Foreign Application Priority Data
Dec. 28, 2023 (CN) 202311854599.0
Dec. 28, 2023 (CN) 202311862038.5

(51) Int. Cl. A61M 5/46; A61B 18/00; A61B 18/14; A61M 5/158; A61N 1/40

(52) U.S. Cl. CPC A61M 5/46; A61B 18/1477; A61M 5/158; A61N 1/403; A61B 2018/00464; A61B 2018/00577

(57) ABSTRACT

Disclosed are a method for controlling an electrical treatment device with injection function and an electrical treatment device. The method includes: driving ends of a plurality of needle bodies to penetrate at least one target depths of a skin tissue by a actuator, and controlling the plurality of the needle bodies to electrically connect to an electric energy output assembly to release an electric energy at least one target depths of the skin tissue, and/or controlling the plurality of the needle bodies to connect to a liquid storage unit to perform drug injection based on at least one target depths of the skin tissue.

### CROSS-REFERENCE TO RELATED APPLICATIONS

This application is a continuation application of International Application No. PCT/CN2024/122903, filed on Sep. 30, 2024, which claims priority to Chinese Patent Application No. 202311854599.0, filed on Dec. 28, 2023 and Chinese Patent Application No. 202311862038.5, filed on Dec. 28, 2023, the entirety of which are herein incorporated by reference.

### TECHNICAL FIELD

The present application relates to the technical field of medical treatment including skin tissue treatment using electrical energy, and in particular to a method for controlling an electrical treatment device with injection function and an electrical treatment device.

### BACKGROUND

With the rapid development of the medical beauty industry, the radio frequency (RF) treatment and injection treatment have become choices of more and more people for aesthetic skin tissue treatment. The treatment delivers the radio frequency energy directly to dermis via microneedles penetrating the skin tissue to a target depth while generating electrical energy, thereby achieving the effect of stimulating the production of collagen to tighten the skin tissue, and the ablation because of heating the target tissue. The injection treatment is to insert needle bodies into the skin tissue to a target depth, to inject a nutritious substance into the skin tissue by the needle bodies, this type of treatment provides drug to skin tissue and may make skin tissue glow, therefore achieving the purpose of cosmetic treatment.

The existing radiofrequency treatment and drug injection treatment generally use radiofrequency microneedles and needle bodies to penetrate the skin tissue for treatment. When performing the radiofrequency and injection treatment, different medical devices need to be used to pierce the skin tissue repetitively multiple times for treatment. The treatment process is cumbersome and the treatment efficiency is low, which affects the user experience.

The above content is only used to assist in understanding the technical solution of the present application and does not mean that the above content is recognized as prior art.

### SUMMARY

The main purpose of the present application is to provide a method for controlling an electrical treatment device with injection function and an electrical treatment device, aiming to solve the technical problem that radiofrequency microneedles and needle bodies need to be used to penetrate the skin tissue repetitively multiple times for treatment in the prior art, and the treatment process is cumbersome and the treatment efficiency is low.

To achieve the above purpose, the present application provides a method for controlling an electrical treatment device with an injection function, including following steps:
driving ends of a plurality of needle bodies to penetrate at least one target depths of a skin tissue by an actuator; and
controlling the plurality of the needle bodies to electrically connect to an electric energy output assembly to release an electric energy at least one target depths of the skin tissue, and/or controlling the plurality of the needle bodies to connect to a liquid storage unit to perform drug injection based on at least one target depths of the skin tissue.

In an embodiment, the plurality of the needle bodies include at least one or more hollow needle bodies and at least one or more solid needle bodies; the hollow needle bodies are capable of being connected to the electric energy output assembly and the liquid storage unit, and the solid needle bodies are capable of being connected to the electric energy output assembly; and
said controlling the plurality of needle bodies to electrically connect to the electric energy output assembly to release an electric energy at least one target depths of the skin tissue; and/or controlling the plurality of the needle bodies to connect to the liquid storage unit to perform drug injection at least one target depths of the skin tissue further includes:
determining one or more selected modes of the hollow needle bodies and the solid needle bodies; and
controlling the hollow needle bodies to release the electrical energy and perform injection, and controlling the solid needle bodies to release the electrical energy based on the one or more selected modes.

In an embodiment, the one or more selected modes include a first work mode, the electrical energy includes radiofrequency energy, and said controlling the hollow needle bodies to release the electrical energy and perform injection based on the one or more selected modes includes:
in response to the one or more selected modes being selected in the first work mode, controlling the hollow needle bodies to release the electrical energy; the first work mode is that the hollow needle bodies release the electrical energy first and then perform injection.

In an embodiment, the one or more selected modes further include a second work mode; said controlling the hollow needle bodies to release electrical energy and perform injection based on the one or more selected modes includes:
in response to the one or more selected modes being selected in the second work mode, controlling the hollow needle bodies to perform injection; the second work mode is that the hollow needle bodies perform injection first and then release the electrical energy.

In an embodiment, the hollow needle bodies and the solid needle bodies are disposed at intervals, and a length of each of the hollow needle bodies is different from a length of each of the solid needle bodies; said driving ends of the plurality of the needle bodies to penetrate at least one target depths of the skin tissue by the actuator includes:
driving the ends of the hollow needle bodies and the ends of the solid needle bodies to penetrate the skin tissue by the actuator simultaneously.

In an embodiment, the actuator is connected to the hollow needle bodies, the solid needle bodies and the processor respectively, and the processor is configured to control the hollow needle bodies and the solid needle bodies to move by the actuator; said driving ends of the plurality of the needle bodies to penetrate at least one target depths of the skin tissue by the actuator includes:
driving the ends of the hollow needle bodies and the ends of the solid needle bodies to penetrate a first target depth of the skin tissue by the actuator;
said controlling the hollow needle bodies to release the electrical energy and perform injection, and controlling the solid needle bodies to release the electrical energy based on the one or more selected modes includes:
controlling the hollow needle bodies to release the electrical energy and perform injection at the first target depth, and controlling the solid needle bodies to release the electrical energy based on the one or more selected modes;
in response to completing the releasing of the electrical energy and completing the injection, driving the hollow needle bodies to move from the first target depth to a second target depth of the skin tissue by the actuator; the first target depth is greater than the second target depth; and
controlling the hollow needle bodies to release the electrical energy and perform the injection at the second target depth, and controlling the solid needle bodies to release the electrical energy based on the one or more selected modes.

In an embodiment, after said controlling the hollow needle bodies to release the electrical energy and perform the injection at the second target depth, and controlling the solid needle bodies to release the electrical energy based on the one or more selected modes, the method further includes:
driving the solid needle bodies to continue to move to a third target depth of the skin tissue by the actuator; the third target depth is greater than the first target depth and the second target depth; and
controlling the solid needle bodies to release the electrical energy in modes with preset parameters during a movement; the modes include a continuous release mode and a fractional release mode.

In an embodiment, the driving ends of the plurality of the needle bodies to penetrate at least one target depths of the skin tissue by the actuator includes:
driving the ends of the hollow needle bodies and the ends of the solid needle bodies to penetrate the first target depth of the skin tissue and/or the second target depth of the skin tissue by the actuator;
said controlling the hollow needle bodies to inject and controlling the solid needle bodies to release electrical energy includes:
controlling the hollow needle bodies to inject in the first target depth and/or the second target depth, and controlling the solid needle bodies to release the electrical energy;
driving the solid needle bodies to continue to move to the third target depth by the actuator; and
controlling the solid needle bodies to release the electrical energy in the preset parameter release mode during the movement.

In an embodiment, the plurality of the needle bodies include at least one or more hollow needle bodies, opposite ends of the plurality of the hollow needle bodies are provided on the needle seat, and the program for controlling the electrical treatment device is configured to implement the following steps:
driving the ends of the hollow needle bodies to penetrate at least one depths of the skin tissue by the actuator;
determining one or more selected modes corresponding to the hollow needle bodies; and
controlling the hollow needle bodies to connect to the electric energy output assembly to release the electric energy and/or to connect to the liquid storage unit to perform injection based on the one or more selected modes.

In an embodiment, the actuator is connected to the needle seat and the processor respectively, and the needle seat is driven to move back and forth by the process controls the actuator; said driving the ends of the plurality of the needle bodies to penetrate at least one target depths of the skin tissue by the actuator includes:
driving the ends of the hollow needle bodies to penetrate a first target depth of the skin tissue of the skin tissue by the actuator;
said controlling the hollow needle bodies to connect to the electric energy output assembly to release the electric energy and/or to connect to the liquid storage unit to perform injection based on the one or more selected modes includes:
controlling the hollow needle bodies to release the electrical energy and/or perform injection in the first target depth based on the one or more selected modes;
driving the hollow needle bodies to move from the first target depth to a second target depth of the skin tissue by the actuator; the first target depth is greater than the second target depth, and at least one the second target depths are provided; and
controlling the hollow needle bodies to release the electrical energy and/or perform injection in the second target depth based on the one or more selected modes.

In an embodiment, said driving the ends of the hollow needle bodies to penetrate at least one depths of the skin tissue by the actuator includes:
driving the hollow needle bodies to penetrate a third target depth of the skin tissue by the actuator, the third target depth is greater than the first target depth and the second target depth;
said controlling the hollow needle bodies to release the electrical energy and/or perform injection based on the one or more selected modes comprises:
controlling the hollow needle bodies to release the electrical energy in the third target depth based on the one or more selected modes;
driving the hollow needle bodies to move from the third target depth to the first target depth and the second target depth respectively by the actuator; and
during the movement, controlling the hollow needle bodies to release the electrical energy in modes with preset parameters based on the one or more selected modes, and/or controlling the hollow needle bodies to perform at least one-shot injection at the first target depth, the second target depth, and during a process of moving from the first target depth to the second target depth; the modes comprise a continuous release mode and a fractional release mode.

In an embodiment, the method further includes:
during the movement from the third preset depth to the first preset depth, controlling the hollow needle bodies to release the electrical energy and/or perform drug injection with the preset parameters, and controlling the hollow needle bodies to perform at least one-shot injection at the first preset depth, the second preset depth, and during the movement from the first preset depth to the second preset depth.

In an embodiment, the method for controlling the electrical treatment device includes:
driving the ends of the plurality of hollow needle bodies to penetrate at least one target depths of the skin tissue by the actuator; and
controlling the hollow needle bodies to release the electrical energy, or perform injection, or release the electrical energy and perform injection at the target preset depth based on the one or more selected modes.

In an embodiment, the method for controlling the electrical treatment device includes:
driving the ends of the plurality of hollow needle bodies to penetrate at least one target depths of the skin tissue by the actuator; and
controlling the hollow needle bodies to release the electrical energy firstly and then perform injection.

In an embodiment, the method for controlling the electrical treatment device includes:
driving the ends of the plurality of hollow needle bodies to penetrate at least one target depths of the skin tissue by the actuator; and
controlling the hollow needle bodies to perform injection.

In an embodiment, the plurality of the needle bodies include at least one or more hollow needle bodies and at least one or more solid needle bodies; the hollow needle bodies are capable of being connected to the liquid storage unit, and the solid needle bodies are capable of being connected to the electric energy output assembly; and
said controlling the plurality of the needle bodies to electrically connect to the electric energy output assembly to release the electric energy at least one target depths of the skin tissue; and/or controlling the plurality of the needle bodies to connect to the liquid storage unit to perform drug injection at least one target depths of the skin tissue further includes:
determining one or more selected modes of the hollow needle bodies and the solid needle bodies; and
controlling the hollow needle bodies to perform injection, and controlling the solid needle bodies to release the electrical energy based on the one or more selected modes.

In an embodiment, the actuator includes a vacuum pump inside the electrical treatment device connecting with a negative pressure pipe inside the tip, which is in fluid communication with a through hole opened on a front surface of the tip, for exerting attraction onto the treatment surface of the skin tissue towards the plurality of the needle bodies; said driving ends of the plurality of the needle bodies to penetrate at least one target depths of the skin tissue by the actuator includes:
driving ends of the hollow needle bodies to penetrate at least one depths of the skin tissue by the vacuum pump based on selected negative pressure modes.

In an embodiment, the actuator includes a first actuating motor configured for driving the needle bodies to move and a second actuating motor configured for injecting the drug from the liquid storage unit into the skin tissue; said driving ends of the plurality of the needle bodies to penetrate at least one target depths of the skin tissue by the actuator includes:
driving the ends of the needle bodies to penetrate the skin tissue by the first actuating motor; and
said controlling the plurality of needle bodies to electrically connect to the electric energy output assembly to release the electric energy and/or to connect to the liquid storage unit to perform drug injection comprises:
controlling the needle bodies to release the electric energy based on the one or more selected modes, and/or controlling the needle bodies to inject by the second actuating motor;
In addition, to achieve the above purpose, the present application further proposes an electrical treatment device, including:
a plurality of hollow needle bodies, a needle seat, an electric energy output assembly, a liquid storage unit, an actuator, a memory, a processor, and a program for controlling the electrical treatment device stored in the memory and executable on the processor; opposite ends of the plurality of the hollow needle bodies being provided on the needle seat, and the actuator being connected to the needle seat and the processor respectively; the processor is further configured for:
driving the ends of the hollow needle bodies to penetrate at least one target depths of the skin tissue by the actuator; and
controlling the hollow needle bodies to electrically connect to the electric energy output assembly to release the electric energy and/or to connect to the liquid storage unit to perform drug injection based on the one or more selected modes.

In addition, to achieve the above purpose, the present application further proposes an electrical treatment device, including: a plurality of needle bodies, a needle seat, an electric energy output assembly, a liquid storage unit, an actuator, a memory, a processor, and a program for controlling the electrical treatment device stored in the memory and executable on the processor; opposite ends of the plurality of needle bodies being provided on the needle seat; the processor is configured for:
driving the ends of the needle bodies to penetrate at least one depths of the skin tissue by the actuator; and
controlling a plurality of the hollow needle bodies to connect to the liquid storage unit to perform injection and/or controlling a plurality of solid needle bodies to electrically connect to the electric energy output assembly for releasing electric energy.

The present application discloses that controlling opposite end of hollow needle bodies and opposite end of solid needle bodies to penetrate the skin tissue by the actuator, the hollow needle bodies are configured for injection, and the solid needle bodies are configured for releasing electrical energy; controlling the hollow needle bodies to perform injection, and controlling the solid needle bodies to release electrical energy. Compared with the prior art, which requires respectively using radiofrequency microneedles and needle bodies to penetrate the skin tissue for multiple times for treatment, the treatment process is relatively cumbersome; since this embodiment can perform radiofrequency treatment by solid needle bodies, and perform injection treatment by hollow needle bodies, thus simplifying the treatment process, reducing the number of times the needle body structure being inserted into the skin tissue, and improving the treatment efficiency and the user's treatment experience.

The present application discloses that driving opposite end of hollow needle bodies to penetrate the skin tissue by the actuator, the hollow needle bodies are configured for releasing electrical energy and/or performing injection; determining the one or more selected modes corresponding to the hollow needle body; controlling the hollow needle bodies to release electrical energy and performing injection based on the one or more selected modes. Compared with the prior art, which requires penetrating the skin tissue repeatedly for multiple times with the needle bodies, the treatment process is relatively cumbersome; since in one embodiment the hollow needle body can be used for radiofrequency treatment and injection treatment both, thus simplifying the treatment process, reducing the number of times the needle body being inserted into the skin tissue, and improving the treatment efficiency and the user's treatment experience.

### BRIEF DESCRIPTION OF THE DRAWINGS

FIG. 1 is a structural schematic view of a control device of an electrical treatment device in hardware operating environment according to an embodiment of the present application.

FIG. 2 is a flow chart of a method for controlling an electrical treatment device according to a first embodiment of the present application.

FIG. 3 is a structural schematic view of a treatment tip of an electrical treatment device in the method for controlling the electrical treatment device according to the present application.

FIG. 4 is a flow chart of the method for controlling the electrical treatment device according to a second embodiment of the present application.

FIG. 5 is a structural schematic view of a control device of an electrical treatment device in a hardware operating environment according to an embodiment of the present application.

FIG. 6 is a flow chart of a method for controlling an electrical treatment device according to a third embodiment of the present application.

FIG. 7 is a structural schematic view of a treatment tip of an electrical treatment device in the method for controlling the electrical treatment device according to the present application.

FIG. 8 is a flow chart of a method for controlling an electrical treatment device according to a fourth embodiment of the present application.

FIG. 9 is a flow chart of a method for controlling an electrical treatment device according to an embodiment of the present application.

The realization of the purpose, functional features and advantages of the present application will be further explained in conjunction with the embodiments and with reference to the accompanying drawings.

### DETAILED DESCRIPTION OF THE EMBODIMENTS

It should be understood that the specific embodiments described herein are only used to explain the present application and are not used to limit the present application.

The technical solutions in the embodiments according to the present application will be clearly and completely described below in conjunction with the accompanying drawings in the embodiments according to the present application. Obviously, the described embodiments are only a part of the embodiments according to the present application, and not all of the embodiments. Based on the embodiments in the present application, all other embodiments obtained by those skilled in the art without making creative labor fall within the scope of the present application.

In the embodiment of the present application, the descriptions of “first”, “second” or the like are only for descriptive purposes and cannot be understood as indicating or implying the relative importance or implicitly indicating the quantity of the technical features indicated. Therefore, features defined as “first” and “second” may explicitly or implicitly include at least one of these features. In addition, the meaning of “and/or” appearing in the entire text includes three parallel solutions, taking “A and/or B” as an example, it includes solution A, or solution B, or a solution that satisfies both A and B at the same time. In addition, the technical solutions of various embodiments can be combined with each other, but it is based on that those of ordinary skill in the art can realize. When the combination of technical solutions is contradictory or cannot be realized, it should be considered that such combination of technical solutions does not exist and is not within the protection scope claimed by the present application.

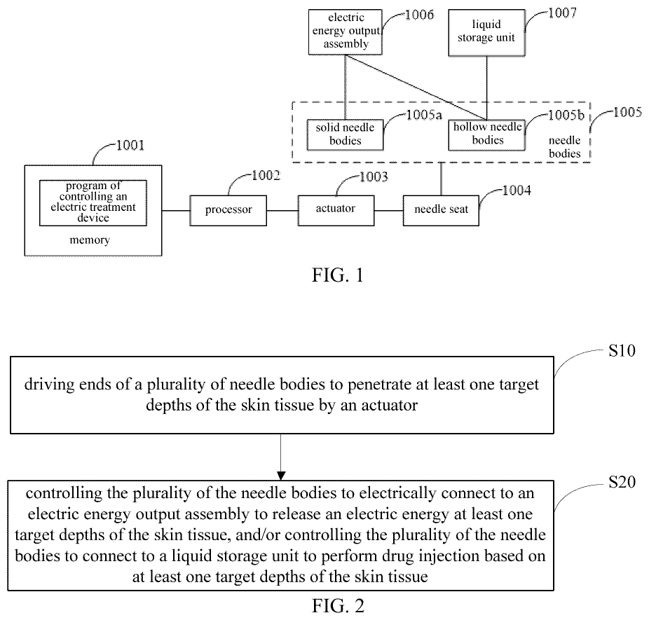

Referring to FIG. 1, FIG. 1 is a structural schematic view of a control device of an electrical treatment device in a hardware operating environment according to an embodiment of the present application.

As shown in FIG. 1, the control device of the electrical treatment device may include a memory 1001, a processor 1002, such as a central processing unit (CPU), an actuator 1003, a needle seat 1004, and a needle body 1005. The memory 1001 may be a high-speed random access memory (RAM) or a stable non-volatile memory (NVM), such as a disk memory.

The structure shown in FIG. 1 does not constitute a limitation on the control device of the electrical treatment device, and may include more or less components than shown in the figure, or a combination of certain components, or differently arranged components.

As shown in FIG. 1, the memory 1001 as a storage medium may include a program for controlling the electrical treatment device.

In the control device of the electrical treatment device shown in FIG. 1, the memory 1001 and the processor 1002 in the control device of the electrical treatment device of the present application can be provided in the control device of the electrical treatment device. The control device of the electrical treatment device calls the program for controlling the electrical treatment device stored in the memory 1001 by the processor 1002, and executes the method for controlling the electrical treatment device provided in the embodiment of the present application.

The embodiment of the present application provides a method for controlling an electrical treatment device. Referring to FIG. 2, FIG. 2 is a flow chart of a method for controlling an electrical treatment device according to the first embodiment of the present application.

In this embodiment, the method for controlling the electrical treatment device is applied to the control device of the electrical treatment device, the device includes a plurality of needle bodies 1005, a needle seat 1004, an actuator 1003, a memory 1001, a processor 1002 and a program for controlling the electrical treatment device stored in the memory 1001 and executable on the processor; the program for controlling the electrical treatment device is configured to implement following steps.

Step S10, driving ends of a plurality of needle bodies to penetrate at least one target depths of the skin tissue by an actuator.

The method in this embodiment is executed by a control device that controls the electrical treatment device to perform radiofrequency treatment and drug injection on the patient, or a control system of other electrical treatment devices that can achieve the same or similar functions and include the control device. The control device of the electrical treatment device (hereinafter referred to as the device) is used to specifically describe the method for controlling the electrical treatment device provided in this embodiment and the following embodiments.

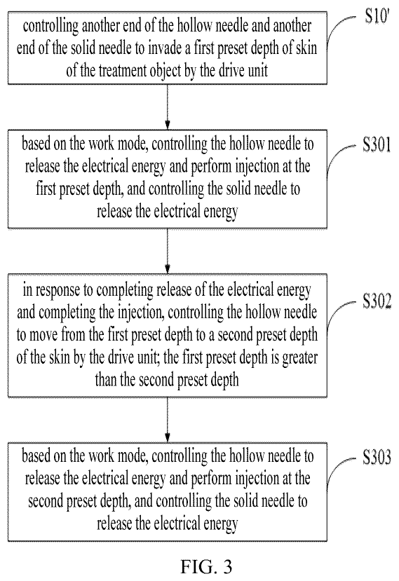

In some embodiments, at least one or more needle bodies 1005 include hollow needle bodies 1005a and solid needle bodies 1005b which are fixed on the needle seat 1004. The drug is a medicine, a moisturizer, a lotion, or any combination thereof. Referring to FIG. 3, FIG. 3 is a structural schematic view of a treatment tip of the device in the method for controlling the device according to the present application. As shown in FIG. 3, the present application can achieve radiofrequency treatment by driving the solid needle bodies 1005a provided on the needle seat that is provide inside the treatment tip to penetrate the skin tissue while release electrical energy, and control the hollow needle bodies 1005b provided on the needle seat that is provided inside the treatment tip to penetrate the skin tissue while performing drug injection.

The above-mentioned actuator can be a structure for directly driving the needle seat to move while penetrating the skin tissue with the needle bodies provided on the needle seat. In this embodiment, the actuator can include but is not limited to a motor, that is, the needle bodies 1005 can be controlled by the motor to penetrate the skin tissue.

The solid needle bodies 1005b in this embodiment may be coupled with an electric energy output assembly (such as a radiofrequency power supply), so that electrical energy can be generated after penetrating the skin tissue, thereby ablating fat, spots, stimulating the production of collagen, and achieving the purpose of radiofrequency treatment. At the same time, since the interior of the hollow needle bodies 1005a are provided with a hollow channel, the hollow channel of the needle bodies may be connected to the liquid storage unit, so that after penetrating the skin tissue, the drug stored in the liquid storage unit can be controlled to be injected into the skin tissue via the hollow channel, thereby achieving the purpose of injection treatment.

To be understood, the drug includes solutions, compound solutions, suspensions, gels, etc.; specific ingredients such as hyaluronic acid, collagen, regenerative microspheres, biological factors, amino acids, vitamins, peptides, high molecular weight polysaccharides or their salts and other nutrients that improve skin conditions.

In this embodiment, the actuator (such as a motor) can be used to control the needle bodies 1005 to release the drug to perform injection treatment. The actuator for controlling the needle bodies 1005 to penetrate and the actuator for controlling the needle bodies 1005 to release the drug can be the same actuator or different actuators.

Each needle body has one end for penetrating the skin tissue and an opposite end for being provided on the needle seat 1004, the actuator may move the ends of the needle bodies between the retracted configuration and the extended configuration. During the treatment process of the skin tissue, the needle seat 1004 is be driven by the actuator to move the ends of the needle bodies to penetrate the skin tissue.

Step S20, controlling the plurality of the needle bodies 1005 to electrically connect to an electric energy output assembly to release an electric energy at least one target depths of the skin tissue, and/or controlling the plurality of the needle bodies 1005 to connect to a liquid storage unit to perform drug injection based on at least one target depths of the skin tissue.

In actual application, when both the hollow needle bodies 1005a and the solid needle bodies 1005b penetrate the skin tissue, it can be that controlling the hollow needle bodies 1005a to inject, so as to perform injection treatment on the skin tissue, and controlling the solid needle bodies 1005b to release electrical energy, so as to perform radiofrequency treatment on the skin tissue.

Furthermore, in addition to performing injection, the hollow needle bodies 1005a can further release electrical energy for radiofrequency treatment. Specifically, the plurality of needle bodies 1005 include at least one or more hollow needle bodies 1005a and at least one or more solid needle bodies 1005b; the hollow needle bodies 1005a are connected to the electric energy output assembly 1006 and the liquid storage unit 1007 both, and the solid needle bodies 1005b are connected to the electric energy output assembly 1006, and the hollow needle bodies 1005a are further configured to release electrical energy. The step of controlling the plurality of needle bodies 1005 to electrically connect to the electric energy output assembly to release the electric energy and/or to connect to the liquid storage unit to perform medicine injection further includes: determining one or more selected modes of the hollow needle bodies 1005b; and based on the one or more selected modes, controlling the hollow needle bodies 1005b to release electrical energy and perform injection, and controlling the solid needle bodies 1005a to release electrical energy.

In this embodiment, the above-mentioned one or more selected modes can be one or more selected modes for performing radiofrequency treatment and injection treatment on the skin tissue. In this embodiment, the one or more selected modes can include a first work mode and a second work mode. The first work mode can be a work mode for performing radiofrequency treatment first and then performing injection treatment, that is, in this work mode, the hollow needle bodies 1005b can release electrical energy firstly and then perform injection. The second work mode can be a work mode for performing injection treatment first and then performing radiofrequency treatment, that is, in this work mode, the hollow needle bodies 1005b can perform injection firstly then release electrical energy.

Correspondingly, step S30 includes:

Step S30′, controlling the hollow needle bodies to release the electrical energy and perform injection, and controlling the solid needle bodies to release the electrical energy based on the one or more selected modes.

When the needle bodies 1005 are connected to the electric energy output assembly, the electrical energy may be conducted from the needle bodies 1005 to other needle bodies on the needle seat by the drug, thereby causing a short circuit and affecting the effect of radiofrequency treatment. Therefore, in order to avoid affecting the effect of radiofrequency treatment, the present embodiment can perform radiofrequency treatment and injection treatment separately or alternatively. Specifically, it can use the work mode of performing radiofrequency treatment firstly and then performing injection treatment, or the work mode of performing injection treatment firstly and then performing radiofrequency treatment to control the needle bodies 1005 for treatment.

Specifically, when the work mode is in the first work mode, the controlling the hollow needle bodies to release electrical energy and perform injection based on the work mode includes: in response to the work mode being the first work mode, controlling the hollow needle to release electrical energy; the first work mode is that the hollow needle bodies release the electrical energy first and then perform injection; when completing release of the electrical energy, controlling the hollow needle to inject.

The electrical energy in this embodiment may include electrical energy, releasing electrical energy in the skin tissue can achieve a tissue thermal effect and stimulate the regeneration effect of collagen during the self-healing of the tissue. Therefore, this embodiment can achieve radiofrequency treatment of the skin tissue by releasing electrical energy in the skin tissue.

In this embodiment, if the work mode is in the first work mode, it needs to perform radiofrequency treatment first, and then perform injection treatment. At this time, it can be that controlling the hollow needle bodies 1005b to release electrical energy to perform radiofrequency treatment on the skin tissue; when completing the radiofrequency treatment, controlling the hollow needle bodies 1005b to inject the drug into the skin tissue to perform injection treatment.

In addition, when the work mode is in the second work mode, the controlling the hollow needle bodies 1005b to release electrical energy and perform injection based on the work mode includes: in response to the one or more selected modes being selected in the second work mode, controlling the hollow needle bodies to perform injection, and the second work mode is that the hollow needle bodies 1005b perform injection firstly and then release the electrical energy; when completing the injection, controlling the hollow needle bodies 1005b to release electrical energy.

In this embodiment, if the work mode is in the second work mode, it needs to perform injection treatment firstly, and then perform radiofrequency treatment. At this time, it can be that controlling the hollow needle bodies 1005b to inject the drug into the skin tissue to perform injection treatment; when completing the injection treatment, controlling the hollow needle bodies 1005b to release electrical energy to perform radiofrequency treatment.

Furthermore, in order to simultaneously meet the two different treatment depths of radiofrequency treatment and injection treatment, and improve the uniformity of radiofrequency treatment and injection treatment simultaneously, the hollow needle bodies 1005b and the solid needle bodies 1005a are disposed at intervals, and a length of each of the hollow needle bodies 1005b is different from a length of each of the solid needle bodies 1005a; the step S10 includes: driving the ends of the hollow needle bodies and the ends of the solid needle bodies to penetrate the skin tissue by the actuator simultaneously.

In this embodiment, the hollow needle bodies and the solid needle bodies can be disposed at intervals provided on the needle seat, so that the radiofrequency treatment and injection treatment can be uniformly performed. At the same time, since the treatment depth of injection treatment and the treatment depth of radiofrequency treatment are different, it is necessary to set the depths of the hollow needle bodies 1005b invading the skin tissue and the depths of the solid needle bodies 1005a invading the skin tissue to be different. In this embodiment, the length of the hollow needle and the length of the solid needle can be different, so that the ends of the hollow needle bodies 1005b and the ends of the solid needle bodies 1005a can be simultaneously driven by the actuator to penetrate the skin tissue at different depths, thereby meeting the treatment depth of radiofrequency treatment and the treatment depth of injection treatment, and improving the effect of radiofrequency treatment and the effect of injection treatment.

This embodiment discloses that driving the ends of the hollow needle bodies 1005b and the ends of the solid needle bodies 1005a to penetrate a skin tissue by the actuator, the hollow needle bodies 1005b are configured for injection, and the solid needle bodies 1005a are configured for releasing electrical energy; controlling the hollow needle bodies 1005b to perform injection, and controlling the solid needle bodies 1005a to release electrical energy. The treatment process requires penetrating the skin tissue repeatedly for multiple time with the needle bodies, since this embodiment can perform radiofrequency treatment by the solid needle bodies 1005a, and perform injection treatment by the hollow needle bodies 1005b, thus simplifying the treatment process, reducing the times of penetrating the skin tissue with the needle bodies, and improving the treatment efficiency and the user's treatment experience.

In an alternative embodiment, step S30 includes:

Step S30: determining one or more selected modes of the hollow needle bodies and the solid needle bodies; and controlling the hollow needle bodies to perform injection, and controlling the solid needle bodies to release the electrical energy based on the one or more selected modes.

In this embodiment, the plurality of the needle bodies 1005 include at least one or more hollow needle bodies 1005b and at least one or more solid needle bodies 1005a; the hollow needle bodies 1005b are capable of being connected to the liquid storage unit, and the solid needle bodies 1005a are capable of being connected to the electric energy output assembly. Under this circumstance, based on the selected modes by the user, the hollow needle bodies is only used to perform injection, as well as the solid needle bodies 1005a are only used to release the electricity energy in each mode, such as the radiofrequency energy.

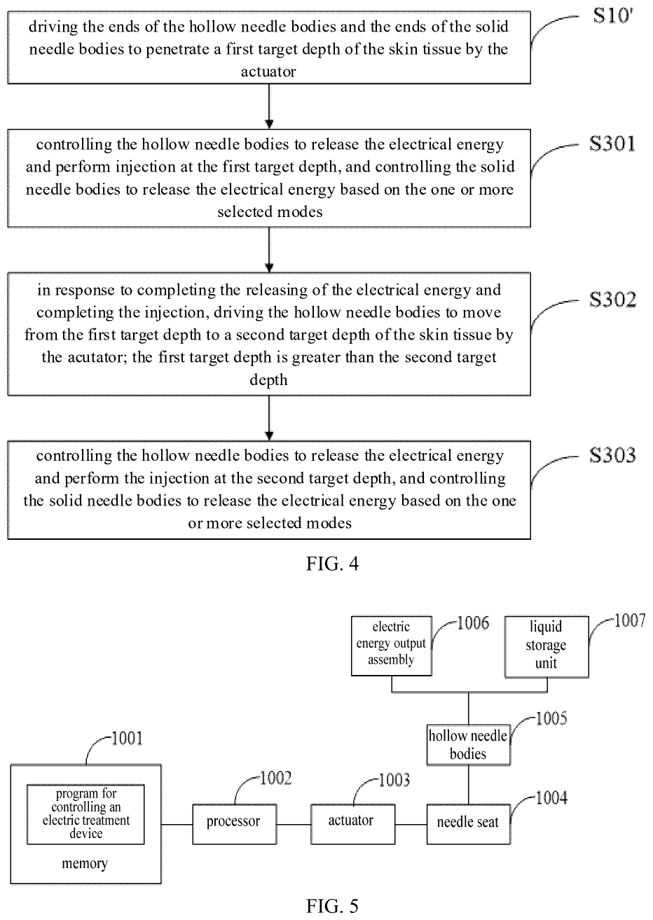

Referring to FIG. 4, FIG. 4 is a flow chart of the method for controlling the electrical treatment device according to a second embodiment of the present application.

Based on the above first embodiment, in order to meet the requirements of different treatment depths in the dermis layer, in this embodiment, the actuator 1003 is connected to the hollow needle bodies 1005b, the solid needle bodies 1005a and the processor 1002 respectively, and the processor 1002 is configured to control the hollow needle bodies 1005b and the solid needle bodies 1005a to move by the actuator 1003; the step S10 includes:

Step S10′, driving the ends of the hollow needle bodies and the ends of the solid needle bodies to penetrate a first target depth of the skin tissue by the actuator.

In this embodiment, the above first target depth can be any depth under the epidermis of the skin tissue. The human skin tissue is divided into the epidermis layer, the dermis layer, the fat layer and the fascia layer from the epidermis to the inside, and the dermis layer in the skin tissue includes the papillary layer and the reticular layer inward in sequence. In this embodiment, the first target depth can be any depth in the dermis layer, the fat layer and the fascia layer.

Correspondingly, step S30 includes:

Step S301, controlling the hollow needle bodies to release the electrical energy and perform injection at the first target depth, and controlling the solid needle bodies to release the electrical energy based on the one or more selected modes.

During treatment, the device can control the treatment ends of the hollow needle bodies 1005b and the treatment ends of the solid needle bodies 1005a by a motor to penetrate the skin tissue. After the hollow needle bodies and the solid needle bodies penetrate the dermis layer of the skin tissue, it can be that controlling the hollow needle bodies based on the one or more selected modes, so as to control the needle bodies to release electrical energy and perform injection in the dermis layer, and controlling the solid needle bodies to release electrical energy, so as to achieve radiofrequency treatment and injection treatment for the skin tissue. Specifically, if the work mode is in the first work mode, it can be that controlling the hollow needle bodies to release electrical energy in the first target depth to perform radiofrequency treatment first, then controlling the hollow needle bodies 1005b to perform injection to achieve injection treatment, and controlling the solid needle bodies 1005a to release electrical energy to perform radiofrequency treatment simultaneously. If the work mode is in the second work mode, it can be that controlling the hollow needle bodies 1005b to inject in the first target depth first, then controlling the hollow needle bodies 1005b to release electrical energy to perform radiofrequency treatment, and controlling the solid needle bodies 1005a to release electrical energy to perform radiofrequency treatment simultaneously.

Step S302, in response to completing the releasing of the electrical energy and completing the injection, driving the hollow needle bodies to move from the first target depth to a second target depth of the skin tissue by the actuator; the first target depth is greater than the second target depth.

In this embodiment, the above-mentioned second target depth can be any depth in the human skin tissue that is less than the first target depth, that is, the second target depth can be any depth in the dermis layer, fat layer and fascia layer that is less than the first target depth. In practical applications, if the first target depth is any depth in the reticular layer in the dermis layer, then the second target depth can be any depth in the papillary layer in the dermis layer, or any depth in the reticular layer that is less than the first target depth; if the first target depth is any depth in the papillary layer in the dermis layer, then the second target depth can be any depth in the reticular layer that is less than the first target depth, and this embodiment does not limit this.

Step S303, controlling the hollow needle bodies to release the electrical energy and perform the injection at the second target depth, and controlling the solid needle bodies to release the electrical energy based on the one or more selected modes.

In a specific implementation, after the motor controls the hollow needle bodies 1005b to perform radiofrequency treatment and injection treatment at the first target depth in the skin tissue, it can continue to control the hollow needle bodies 1005b to move from the first target depth to the second target depth, and control the hollow needle bodies 1005b release electrical energy and perform injection at the second target depth based on the first work mode or the second work mode, thus achieving radiofrequency treatment and injection treatment at the second target depth.

Furthermore, since the depth of radiofrequency treatment is different from that the depth of injection treatment, in order to meet the requirements of different treatment depths of radiofrequency treatment and injection treatment, after step S303, the method further includes: driving the solid needle bodies 1005a to continue to move to a third target depth of the skin tissue by the actuator; the third target depth is greater than the first target depth and the second target depth; and controlling the solid needle bodies 1005a to release the electrical energy in modes with preset parameters during a movement; the modes include a continuous release mode and a fractional release mode.

The above-mentioned third target depth can be any depth deeper than the first target depth and the second target depth in the skin tissue, and this embodiment does not limit this. In actual application, the skin tissue is divided into epidermis layer, dermis layer, fat layer and fascia layer from the epidermis to the inside, and the third target depth in this embodiment can be any depth between the fat layer and the fascia layer.

Radiofrequency treatment at different layers in the skin tissue can produce different treatment effects. Therefore, this embodiment can control the solid needle bodies to move to different layers of the skin tissue for radiofrequency treatment by a motor.

When the solid needle bodies 1005a move from the dermis layer to the third target depth in the skin tissue, the electrical energy can be released at the third target depth to perform radiofrequency treatment on the skin tissue. Specifically, when the third target depth is the fat layer in the skin tissue, the electrical energy can be released in the fat layer to decompose and reduce fat, improve microcirculation, promote collagen contraction and denaturation, start the regeneration and reorganization of collagen, repair and reshape, thereby tightening the skin tissue. When the third target depth is the fascia layer in the skin tissue, electrical energy can be released in the fascia layer to contract the fascia. After the relaxed fascia is contracted, the fascia becomes more elastic and provides better support to the skin tissue, thereby improving the problem of skin tissue laxity.

The preset parameter release mode can be the mode that the needle bodies 1005 releases electrical energy. In this embodiment, the preset parameter release mode can include a continuous release mode and a fractional release mode. The continuous release mode can be a mode in which the needle bodies 1005 continuously releases electrical energy in the process of moving from the fascia layer to the fat layer and the dermis layer; the fractional release mode can be a mode in which the needle bodies 1005 release electrical energy in the fascia layer, the fat layer and the dermis layer respectively during the movement.

Furthermore, if the hollow needle bodies 1005b are only capable of injection treatment, step S10 includes: driving the ends of the hollow needle bodies and the ends of the solid needle bodies to penetrate the first target depth of the skin tissue and/or the second target depth of the skin tissue by the actuator.

Correspondingly, step S20 includes: controlling the hollow needle bodies to inject in the first target depth and/or the second target depth, and controlling the solid needle bodies to release the electrical energy; driving the solid needle bodies to continue to move to the third target depth by the actuator; and controlling the solid needle bodies to release the electrical energy in the preset parameter release mode during the movement.

In this embodiment, the hollow needle bodies 1005b can be capable of injection treatment, and the solid needle bodies can be capable of radiofrequency treatment. Specifically, first, it can be that controlling the hollow needle bodies 1005b to penetrate the first target depth and/or a second target depth of the dermis layer, and controlling the hollow needle bodies 1005b to inject at the first target depth and/or the second target depth to achieve injection treatment of the skin tissue; controlling the solid needle bodies 1005a to release electrical energy at the first target depth and/or the second target depth simultaneously, so as to achieve radiofrequency treatment of the skin tissue. When completing the treatment at the first target depth and/or the second target depth, it can be that continuously controlling the solid needle bodies 1005a to move to the fat layer or the fascia layer in the skin tissue, and controlling the solid needle bodies 1005a to release electrical energy in the continuous release mode and the fractional release mode during the movement, so as to perform radiofrequency treatment on the skin tissue in the fat layer and the fascia layer.

Furthermore, in order to relieve the user's pain during the treatment process, the electrical energy is further divided into: medium frequency electrical energy and low frequency electrical energy; the step of controlling the plurality of needle bodies to electrically connect to the electric energy output assembly to release electric energy and/or to connect to the liquid storage unit to perform drug injection includes: controlling the needle bodies to release medium frequency electrical energy or low frequency electrical energy (electrical stimulation). The treatment by using medium frequency electrical energy or low frequency electrical energy may stimulate muscles to relieve pain. The pluralities of needle bodies 1005 include at least one or more hollow needle bodies 1005b, or a combination of at least one or more hollow needle bodies 1005b and at least one or more solid needle bodies 1005a.

Since the needle bodies 1005 releases medium frequency electrical energy or low frequency electrical energy to perform electrical treatment on the skin tissue, which can relieve pain, thus, in this embodiment, when the needle bodies 1005 penetrate the skin tissue or performs injection, the needle bodies 1005 can be controlled to release medium frequency electrical energy or low frequency electrical energy to relieve the user's pain. Both the solid needle bodies 1005a and the hollow needle bodies 1005b can release medium frequency electrical energy or low frequency electrical energy to relieve pain.

In this embodiment, electrical energy and medium frequency electrical energy/low frequency electrical energy can be released alternately, that is, it can be that releasing electrical energy first, and then releasing medium frequency electrical energy/low frequency electrical energy at intervals, and then releasing electrical energy continuously. The release of medium frequency electrical energy/low frequency electrical energy can cover the entire treatment process. In addition, the present solution can set up independent energy sources for electrical energy and medium frequency electrical energy/low frequency electrical energy respectively, and the device can release radiofrequency and medium frequency electrical energy/low frequency electrical energy simultaneously during the treatment process, or can control and switch the energy source by the switch for alternating output.

Specifically, the controlling the needle bodies 1005 to release medium frequency electrical energy or low frequency electrical energy includes: controlling the needle bodies 1005 to output medium frequency electrical energy or low frequency electrical energy in an interval release mode, and the interval release mode is pulse output based on a preset time interval; and/or controlling the needle bodies to output medium frequency electrical energy or low frequency electrical energy simultaneously or alternately at different times.

In the present embodiment, the needle bodies 1005 can output medium frequency electrical energy or low frequency electrical energy in pulses based on a preset time interval, that is, it can output medium frequency electrical energy or low frequency electrical energy in a pulse output mode at regular intervals, and the energy output interval can be preset based on actual treatment, which is not limited in this embodiment. In addition, the needle bodies 1005 can further simultaneously or alternately output medium frequency electrical energy or low frequency electrical energy and electrical energy, that is, it can first output medium frequency electrical energy or low frequency electrical energy, then output electrical energy, and then continue to output medium frequency electrical energy or low frequency electrical energy, and periodically in sequence, thereby achieving radiofrequency treatment of the user and relieving the user's pain simultaneously.

In this embodiment, driving the ends of the hollow needle bodies and the ends of the solid needle bodies to penetrate the first target depth of the skin tissue by the actuator; controlling the hollow needle bodies to release electrical energy and inject in the first target depth, and controlling the solid needle bodies to release electrical energy based on the one or more selected modes. When completing release of the electrical energy and completing the injection, driving the hollow needle bodies to move from the first target depth to the second target depth by the actuator; controlling the hollow needle bodies to release electrical energy and inject in the second target depth, and controlling the solid needle bodies to release electrical energy based on the one or more selected modes, thus enabling to meet the needs of different treatment depths in the dermis layer. At the same time, in this embodiment, it further can control the needle bodies to release medium frequency electrical energy or low frequency electrical energy to relieve the user's pain.

Based on the above-mentioned embodiments of the method for controlling the electrical treatment device, the first embodiment of the electrical treatment device of the present application is proposed.

In this embodiment, the electrical treatment device includes at least one or more needle bodies 1005, a needle seat 1004, an actuator 1003, a memory 1001, a processor 1002, and a program for controlling an electrical treatment device stored in the memory 1002 and executable on the processor. Opposite ends of the plurality of the needle bodies 1005 are provided on the needle seat 1004, and the processor 1002 controls the actuator 1003 to drive the needle seat 1004.

The processor 1002 is configured for driving the ends of the needle bodies 1005 to penetrate at least one depths of the skin tissue by the actuator 1003, the hollow needle bodies 1005b are configured for performing injection, and the solid needle bodies 1005a are configured for releasing electrical energy.

The processor 1002 is further configured for controlling the needle bodies 1005 connected with the liquid storage unit for performing injection and/or to electrically connected with the electric energy output assembly for releasing electrical energy; the plurality of needle bodies 1005 include at least one or more hollow needle bodies 1005b and at least one or more solid needle bodies 1005a.

This embodiment discloses that driving the ends of the hollow needle bodies 1005b and the ends of the solid needle bodies 1005a to penetrate a skin tissue by an actuator, the hollow needle bodies 1005b are capable of performing injection, and the solid needle bodies 1005a are capable of releasing electrical energy; controlling the hollow needle bodies 1005b to perform injection, and controlling the solid needle bodies 1005a to release electrical energy. Compared with the prior art, which requires respectively using radiofrequency microneedles to insert into the skin tissue for multiple times for treatment, the treatment process is relatively cumbersome; since this embodiment can perform radiofrequency treatment by the solid needle bodies, and perform injection treatment by the hollow needle bodies, thus simplifying the treatment process, reducing the number of times the needle body structure being inserted into the skin tissue, and improving the treatment efficiency and the user's treatment experience.

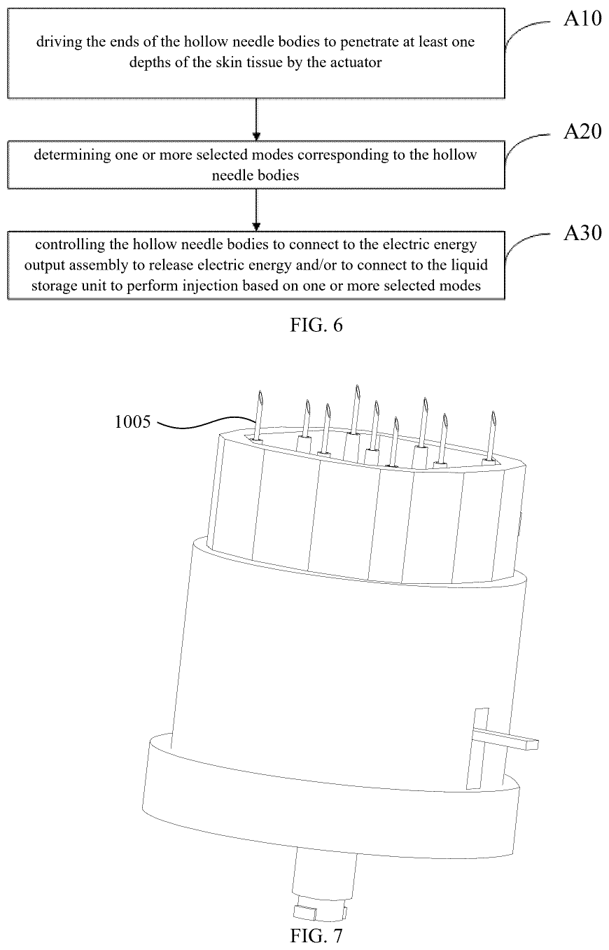

This embodiment of the application further provides a method for controlling an electrical treatment device. Referring to FIG. 6, FIG. 6 is a flow chart of a method for controlling an electrical treatment device according to a third embodiment of the present application.

In this embodiment, the method for controlling the electrical treatment device is applied to the control device of the electrical treatment device, the device includes at least one or more hollow needle bodies 1005, one end of at least one or more the hollow needle bodies 1005b are provided on the needle seat 1004, and the program for controlling the electrical treatment device is configured to implement the following steps:

Step A10, driving the ends of the hollow needle bodies to penetrate at least one depths of the skin tissue by the actuator.

The execution subject of the method of this embodiment may be a control device that controls the electrical treatment device to perform radiofrequency treatment and injection treatment on the user, or a control system of other electrical treatment devices that can achieve the same or similar functions and include the control device. The control device of the electrical treatment device (hereinafter referred to as the device) is used to specifically describe the method for controlling the electrical treatment device provided in this embodiment and the following embodiments.

Referring to FIG. 7, FIG. 7 is a structural schematic view of an electrical treatment device in the method for controlling the electrical treatment device according to the present application. As shown in FIG. 7, the present application can control the hollow needle bodies 1005b in the electrical treatment device to release electrical energy and perform injection, so as to achieve radiofrequency treatment and injection treatment for the user.

The above-mentioned actuator can be a motor for driving the needle seat and then driving the needle bodies provided on the needle seat to move. In this embodiment, the actuator can include but is not limited to a motor, that is, in this embodiment, it can be that controlling the needle body by the motor to penetrate the skin tissue. The skin tissue can be any object that undergoes radiofrequency treatment and injection treatment.

The hollow needle in this embodiment can connect to an electric energy output assembly (such as a radiofrequency power supply), so that electrical energy can be generated after penetrating the skin tissue, thereby ablating fat, spots, stimulating the production of collagen, and achieving the purpose of radiofrequency treatment. In addition, the interior of the hollow needle body is provided with a hollow channel, the hollow channel of the needle body can connect to the liquid storage unit, so that after penetrating the skin tissue, the drug stored in the liquid storage unit can be controlled to be injected into the skin tissue via the hollow channel, thereby achieving the purpose of injection treatment. The drug includes solutions, compound solutions, suspensions, gels, etc.; specific ingredients such as hyaluronic acid, collagen, regenerative microspheres, biological factors, amino acids, vitamins, peptides, high molecular weight polysaccharides or their salts and other nutrients that improve skin tissue conditions.

In this embodiment, the actuator (such as a motor) can be used to inject the drug from the liquid storage unit into the skin tissue through the hollow needle bodies. The actuator is configured for driving the hollow needle bodies to move back and forth, and the actuator (such as motor) is configured for injecting the drug from the liquid storage unit into the skin tissue can be the same actuator or different actuators.

The two ends of the hollow needle body can be a connection end and a treatment end respectively. The connection end of the hollow needle body is connected to the needle seat, and the treatment end can be provided to be suspended. When treating the skin tissue, the needle seat is driven by the motor to move back and forth while driving the treatment ends of the hollow needle bodies to penetrate the skin tissue.

Step A20, determining one or more selected modes corresponding to the hollow needle bodies.

In this embodiment, the above-mentioned one or more selected modes can be a work mode for performing radiofrequency treatment and injection treatment on the skin tissue. In this embodiment, the work mode includes a first work mode and a second work mode. The first work mode is performing radiofrequency treatment first and then performing injection treatment, that is, in this work mode, the hollow needle body 1005b can release electrical energy first and then perform injection. The second work mode is performing injection treatment first and then performing radiofrequency treatment, that is, in this work mode, the hollow needle body can release electrical energy first and then perform injection.

Step A30, controlling the hollow needle bodies to connect to the electric energy output assembly to release the electric energy and/or to connect to the liquid storage unit to perform injection based on the one or more selected modes.

When the hollow needle bodies 1005b are connected to the electric energy output assembly, the electrical energy may be conducted from the hollow needle bodies 1005b to other hollow needle bodies on the needle seat by the drug, thereby causing a short circuit and affecting the effect of radiofrequency treatment. Therefore, in order to avoid affecting the effect of radiofrequency treatment, the present embodiment can perform radiofrequency treatment and injection treatment separately or alternatively. Specifically, it can use the one or more selected modes of performing radiofrequency treatment first and then performing injection treatment, or the one or more selected modes of performing injection treatment first and then performing radiofrequency treatment to control the needle bodies 1005 for treatment.

Specifically, when the work mode is in the first work mode, the step A30 includes: in response to the work mode being in the first work mode, controlling the hollow needle bodies to release electrical energy; the first work mode is a work mode in which the hollow needle bodies release the electrical energy first and then perform injection; when completing release of the electrical energy, controlling the hollow needle bodies to inject.

The electrical energy in this embodiment may include electrical energy, releasing electrical energy in the skin tissue can achieve a tissue thermal effect and stimulate the regeneration effect of collagen during the self-healing of skin tissue. Therefore, this embodiment can achieve radiofrequency treatment of the skin tissue by releasing electrical energy in the skin tissue.

In this embodiment, if the work mode is in the first work mode, it needs to perform radiofrequency treatment first, and then perform injection treatment. At this time, it can be that controlling the hollow needle bodies to release electrical energy to perform radiofrequency treatment on the skin tissue; when completing the radiofrequency treatment, controlling the hollow needle bodies to inject the drug into the skin tissue to perform injection treatment on the skin tissue.

In addition, when the work mode is in the second work mode, the step A30 includes: in response to the work mode being in the second work mode, controlling the hollow needle bodies to perform injection; the second work mode is that the hollow needle bodies performs injection first and then releases the electrical energy; when completing the injection, controlling the hollow needle bodies to release electrical energy.

In this embodiment, if the work mode is in the second work mode, it needs to perform injection treatment first, and then perform radiofrequency treatment. At this time, it can be that controlling the hollow needle bodies to inject the drug into the skin tissue to perform injection treatment on the skin tissue; when completing the radiofrequency injection, controlling the hollow needle bodies to release electrical energy to perform radiofrequency treatment on the skin tissue

This embodiment discloses that driving the ends of the hollow needle bodies to penetrate the skin tissue by an actuator, the hollow needle bodies are configured for releasing electrical energy and/or performing injection; determining the one or more selected modes corresponding to the hollow needle body; controlling the hollow needle bodies to release electrical energy and performing injection based on the one or more selected modes. The treatment process requires penetrating the skin tissue repeatedly for multiple times with needle bodies, in this embodiment, the hollow needle bodies can be both used for applying radiofrequency energy and injecting the drug into the skin tissue, thus simplifying the treatment process, reducing the repeated times of the needle bodies inserted into the skin tissue, and improving the treatment efficiency and the user's treatment experience.

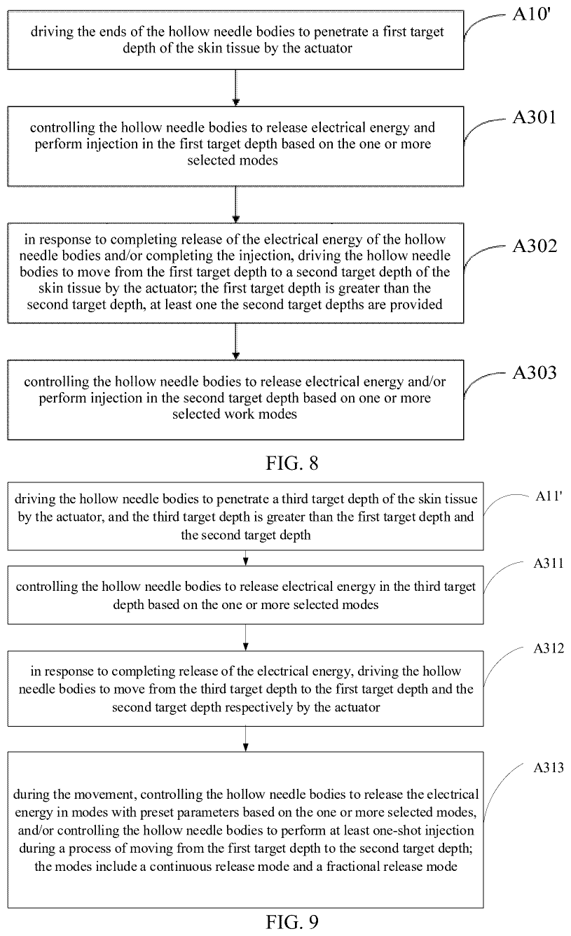

Referring to FIG. 8, FIG. 8 is a flow chart of a method for controlling an electrical treatment device according to a fourth embodiment of the present application.

Based on the above-mentioned third embodiment, in order to meet the requirements of different treatment depths in the dermis layer, the actuator 1003 is connected to the needle seat 1004 and the processor 1002 respectively, and the needle seat 1004 is driven to move by that the processor 1002 controls the actuator 1003. In this embodiment, step A10 includes:

Step A10′, driving the ends of the hollow needle bodies to penetrate a first target depth of the skin tissue by the actuator.

In this embodiment, the above first target depth can be any depth under the epidermis of the skin tissue. The skin tissue is divided into the epidermis layer, the dermis layer, the fat layer and the fascia layer from the epidermis to the inside, and the dermis layer in the skin tissue includes the papillary layer and the reticular layer inward in sequence. In this embodiment, the first target depth can be any depth in the dermis layer, the fat layer and the fascia layer.

Correspondingly, step A30 includes:

Step A301, controlling the hollow needle bodies to release electrical energy and perform injection in the first target depth based on the one or more selected modes.

During treatment, the treatment ends of the hollow needle bodies are driven by a motor to penetrate the skin tissue. After the hollow needle bodies penetrate the dermis layer of the skin tissue, it can be that controlling the hollow needle bodies based on the one or more selected modes, so as to control the hollow needle bodies to release electrical energy and perform injection in the dermis layer, thus achieving radiofrequency treatment and injection treatment for the skin tissue. Specifically, if the work mode is in the first work mode, it can be that controlling the hollow needle bodies to release electrical energy in the first target depth to perform radiofrequency treatment first, then controlling the hollow needle bodies to perform injection to achieve injection treatment. If the work mode is in the second work mode, it can be that controlling the hollow needle bodies to inject in the first target depth first, and then controlling the hollow needle bodies to release electrical energy to perform radiofrequency treatment.

Step A302, in response to completing release of the electrical energy of the hollow needle bodies and/or completing the injection, driving the hollow needle bodies to move from the first target depth to a second target depth of the skin tissue by the actuator; the first target depth is greater than the second target depth, and at least one the second target depths are provided.

In this embodiment, the above-mentioned second target depth can be any depth in the human skin tissue that is less than the first target depth, that is, the second target depth can be any depth in the dermis layer, fat layer and fascia layer that is less than the first target depth. In practical applications, if the first target depth is any depth in the reticular layer in the dermis layer, then the second target depth can be any depth in the papillary layer in the dermis layer, or any depth in the reticular layer that is less than the first target depth; if the first target depth is any depth in the papillary layer in the dermis layer, then the second target depth can be any depth in the reticular layer that is less than the first target depth, and this embodiment does not limit this.

This embodiment does not limit the number of second target depths, that is, there may be multiple second target depths. In practical applications, after moving from the first target depth to the second target depth of the skin tissue and performing treatment at the second target depth, radiofrequency treatment and injection treatment can be performed based on moving the needle bodies to the next second target depth.

Step A303, controlling the hollow needle bodies to release electrical energy and/or perform injection in the second target depth based on the work mode.

In a specific implementation, after the motor controls the hollow needle bodies to perform radiofrequency treatment and injection treatment at the first target depth of the skin tissue, it can continue to control the hollow needle bodies to move from the first target depth to the second target depth, and control the hollow needle bodies release electrical energy and perform injection at the second target depth based on the first work mode or the second work mode, thus achieving radiofrequency treatment and injection treatment at the second target depth.

Furthermore, referring to FIG. 9, since the depth of radiofrequency treatment is different from the depth of injection treatment, in order to meet the requirements of different treatment depths of radiofrequency treatment and injection treatment, A10 includes:

Step A11′, driving the hollow needle bodies to penetrate a third target depth of the skin tissue by the actuator, and the third target depth is greater than the first target depth and the second target depth.

The above-mentioned third target depth can be any depth deeper than the first target depth and the second target depth in the skin tissue, and this embodiment does not limit this. In actual application, the human skin tissue is divided into epidermis layer, dermis layer, fat layer and fascia layer from the epidermis to the inside, and the third target depth in this embodiment can be any depth in the fat layer and the fascia layer.

Correspondingly, step A30 includes:

Step A311, controlling the hollow needle bodies to release electrical energy in the third target depth based on the one or more selected modes.

The radiofrequency treatment at different layers of the skin tissue can generate different treatment effects. Therefore, this embodiment can drive the hollow needle bodies to move to different layers of the skin tissue for radiofrequency treatment by the actuator such as the motor.

When the hollow needle bodies move from the dermis layer to the third target depth in the skin tissue, electrical energy can be released at the third target depth to perform radiofrequency treatment on the skin tissue. Specifically, when the third target depth is the fat layer in the skin tissue, electrical energy can be released in the fat layer to decompose and reduce fat, improve microcirculation, promote collagen contraction and denaturation, start the regeneration and reorganization of collagen, repair and reshape, thereby tightening the skin tissue. When the third target depth is the fascia layer in the skin tissue, electrical energy can be released in the fascia layer to contract the fascia. After the relaxed fascia is contracted, the fascia becomes more elastic and provides better support to the skin tissue, thereby improving the problem of skin tissue laxity.

Step A312, in response to completing release of the electrical energy, driving the hollow needle bodies to move from the third target depth to the first target depth and the second target depth respectively by the actuator.

In this embodiment, after the hollow needle bodies perform radiofrequency treatment in the fat layer and fascia layer in the skin tissue, it can then to move to the first target depth and the second target depth of the dermis layer in the skin tissue for radiofrequency treatment and treatment.

Step A313, during the movement, controlling the hollow needle bodies to release the electrical energy in modes with preset parameters based on the one or more selected modes, and/or controlling the hollow needle bodies to perform at least one-shot injection at the first target depth, the second target depth, and during a process of moving from the first target depth to the second target depth; the modes include a continuous release mode and a fractional release mode.

The preset parameter release mode can be the mode that the hollow needle bodies release electrical energy. In this embodiment, the preset parameter release mode can include a continuous release mode and a fractional release mode. The continuous release mode can be a mode in which the hollow needle bodies continuously release electrical energy in the process of moving from the fascia layer to the fat layer and the dermis layer; the fractional release mode can be a mode in which the hollow needle bodies release electrical energy in the fascia layer, the fat layer and the dermis layer respectively during the movement.

In practical applications, firstly, it can be that driving the hollow needle bodies to penetrate a third target depth by the actuator, that is, any depth in the fat layer and the fascia layer, and adopting the first work mode or the second work mode to control the hollow needle bodies to release electrical energy at any depth in the fat layer and the fascia layer for radiofrequency treatment. When completing the release of the electrical energy, that is, completing the radiofrequency treatment, it can be that controlling the hollow needle bodies to move from the third target depth to the first target depth and the second target depth in the dermis layer respectively, adopting the first work mode or the second work mode to control the hollow needle bodies to release electrical energy in a continuous release mode or a fractional release mode during the moving, and controlling the hollow needle bodies to perform at least one injection at the first target depth target depth, the second target depth and during the process of moving from the first target depth to the second target depth, thereby achieving radiofrequency treatment of the skin tissue in the fascia layer or fat layer, and performing radiofrequency treatment and injection treatment on the skin tissue in the dermis layer.

Furthermore, in order to achieve radiofrequency treatment on the skin tissue in the fascia layer or fat layer, and perform injection treatment on the skin tissue in the dermis layer, after step A312, the method further includes: during a process of moving from the third target depth to the first target depth, controlling the hollow needle bodies to release electrical energy and/or perform drug injection adopting the preset parameter release mode, and controlling the hollow needle bodies to perform at least one injection at the first target depth, the second target depth, and during the process of moving from the first target depth to the second target depth.

In some embodiments, the preset parameters include radiofrequency power, radiofrequency pulse frequency, radiofrequency treatment time, injection drug dosage, injection drug type or the like, which are preset according to the skin tissue conditions and user instructions.

In this embodiment, it can be that driving the hollow needle bodies to move from the third target depth in the skin tissue (that is, any depth in the fascia layer or fat layer) to the first target depth by the actuator, and controlling the hollow needle bodies to release electrical energy in a continuous release mode or a fractional release mode during the process of moving from the third target depth to the first target depth; simultaneously, during the process of the hollow needle bodies moving to the first target depth and the second target depth, and moving from the first target depth to the second target depth, that is, during the process of the hollow needle bodies moving in the dermis layer, controlling the hollow needle bodies to perform at least one shot injection to achieve radiofrequency treatment in the fascia layer or fat layer and injection treatment in the dermis layer.

Furthermore, step A10 further includes: driving the ends of the plurality of needle bodies to penetrate at least one target depths of the skin tissue by the actuator.

The above-mentioned target depth can be any one of the first target depth, the second target depth and the third target depth, that is, the present embodiment does not limit the depth of hollow needle bodies invading the skin tissue, and the hollow needle bodies can penetrate any layer of the skin tissue for radiofrequency treatment and injection treatment.

Correspondingly, step A30 includes: controlling the hollow needle bodies to release the electrical energy, or perform injection, or release the electrical energy and perform injection at the target preset depth based on the one or more selected modes.

In this embodiment, after the hollow needle bodies penetrate the skin tissue to at least one target preset depth, such as the first target depth, the second target depth, or the third target depth, it can be that performing injection alone or releasing electrical energy alone or releasing electrical energy and performing injection simultaneously in these depths. In an alternative embodiment, the method for controlling the electrical treatment device further includes:
driving the ends of the plurality of needle bodies to penetrate at least one target depths of the skin tissue by the actuator; and
controlling the hollow needle bodies to release the electrical energy, or perform injection, or release the electrical energy and perform injection at the target preset depth based on the one or more selected modes.

In an alternative embodiment, the method for controlling the electrical treatment device further includes:
driving the ends of the plurality of needle bodies to penetrate at least one target depths of the skin tissue by the actuator; and
controlling the hollow needle bodies to release the electrical energy or perform injection.

In an alternative embodiment, the method for controlling the electrical treatment device further includes:
driving the ends of the plurality of needle bodies to penetrate at least one target depths of the skin tissue by the actuator; and
controlling the hollow needle bodies to perform injection.

Furthermore, in order to relieve the user's pain during the treatment process, the electrical energy further includes: medium frequency electrical energy or low frequency electrical energy. The method further includes: controlling the hollow needle bodies to release medium frequency electrical energy (electrical stimulation) or low frequency electrical energy, and the medium frequency electrical energy or low frequency electrical energy is configured to stimulate muscles to relieve pain.

Since the hollow needle bodies release medium frequency electrical energy or low frequency electrical energy to perform electrical treatment on the skin tissue, which can relieve pain, thus, in this embodiment, when the hollow needle bodies penetrate the skin tissue or performs injection, the hollow needle bodies can be controlled to release medium frequency electrical energy or low frequency electrical energy to relieve the user's pain.

In this embodiment, electrical energy and medium frequency electrical energy/low frequency electrical energy can be released alternately, that is, it can be that releasing electrical energy first, then releasing medium frequency electrical energy/low frequency electrical energy at intervals, and then releasing electrical energy continuously. The release of medium frequency electrical energy/low frequency electrical energy can cover the entire treatment process. In addition, the present solution can set up independent energy sources for electrical energy and medium frequency electrical energy/low frequency electrical energy respectively, and the device can release radiofrequency and medium frequency electrical energy/low frequency electrical energy simultaneously during the treatment process, or can control and switch the energy source by the switch for alternating output.

Specifically, the controlling the hollow needle bodies to release medium frequency electrical energy or low frequency electrical energy includes: controlling the hollow needle bodies to output medium frequency electrical energy or low frequency electrical energy in an interval release mode, and the interval release mode is pulse output based on a preset time interval; and/or controlling the needle bodies to output medium frequency electrical energy or low frequency electrical energy simultaneously or alternately at different times.

In the present embodiment, the hollow needle bodies can output medium frequency electrical energy or low frequency electrical energy in pulses based on a preset time interval, that is, it can output medium frequency electrical energy or low frequency electrical energy in a pulse output mode at regular intervals, and the present embodiment does not limit the energy output interval. In addition, the hollow needle bodies can further simultaneously or alternately output medium frequency electrical energy or low frequency electrical energy and electrical energy, that is, it can first output medium frequency electrical energy or low frequency electrical energy, then output electrical energy, then continue to output medium frequency electrical energy or low frequency electrical energy, and cycle in sequence, thereby achieving radiofrequency treatment of the user and relieving the user's pain simultaneously.

Furthermore, in order to increase the stability of the hollow needle bodies penetrating the skin tissue, in this embodiment, it can be that controlling the hollow needle bodies to penetrate the skin tissue by selecting negative pressure modes or motor-driving modes. Specifically, the actuator includes a vacuum pump inside the electrical treatment device connecting with a negative pressure pipe inside the treatment tip, which is in fluid communication with a through hole opened on a front surface of the tip, for exerting attraction onto the treatment surface of the skin tissue towards the plurality of the needle bodies; the step of driving the needle seat to control opposite ends of the plurality of needle bodies to penetrate at least one depths of the skin tissue by the actuator includes: driving opposite ends of the needle bodies to penetrate at least one depths of the skin tissue by the vacuum pump based on the selected negative pressure modes, of which, the needle bodies include at least one or more hollow needle bodies, or a combination of at least one or more hollow needle bodies and at least one or more solid needle bodies.

The above-mentioned negative pressure unit may be a device for exerting negative pressure to attract the treatment surface of the skin tissue towards the needle bodies; correspondingly, the above-mentioned negative pressure modes is defined by applying negative pressure to the skin tissue to cause the hollow needle bodies penetrate the skin tissue.

In this embodiment, the actuator may be a vacuum pump for applying negative pressure. When performing radiofrequency treatment and injection treatment on the skin tissue, it can be that applying negative pressure to the skin tissue by the vacuum pump, exerting attraction onto the treatment surface of the skin tissue, whereas, the hollow needle bodies remain stationary, so that allowing the hollow needle bodies to penetrate the skin tissue. The size of the skin tissue deformation can be controlled by adjusting values of the negative pressure, so as to adjust the depths of penetrating the skin tissue with the needle bodies.

Furthermore, the actuator includes a first actuating motor configured for driving the needle bodies to move and a second actuating motor configured for injecting the drug from the liquid storage unit into the skin tissue; the step of driving the needle seat to control opposite ends of the plurality of needle bodies to penetrate at least one depths of the skin tissue by the actuator includes: driving the ends of the needle bodies to penetrate the skin tissue by the first actuating motor.

The step of controlling at least one or more needle bodies to electrically connect to the electric energy output assembly to release electric energy and/or to connect to the liquid storage unit to perform drug injection includes: controlling the needle bodies to release the electric energy based on the one or more selected modes, and/or driving the needle bodies to inject by the second actuating motor.

The above-mentioned first actuating motor and the second actuating motor can both be motors, that is, two motors can be provided in this embodiment, the first motor is configured to drive the hollow needle bodies provided on the needle seat to move back and forth while penetrating the skin tissue, and the second motor is configured to inject the drug from the liquid storage unit into the skin to realize the hollow needle bodies to perform injection, for example the second motor is coupled with a piston and driving the piston to move forth along an inside wall of the liquid storage unit.

In practical applications, when performing treatment on the skin tissue, it can be that controlling the hollow needle bodies to penetrate the skin tissue by the first actuating motor first, then controlling the hollow needle bodies to release electrical energy based on the one or more selected modes corresponding to the hollow needle bodies, and driving the hollow needle bodies to perform injection by the second actuating motor.

In this embodiment, driving opposite ends of the hollow needle bodies to penetrate the skin tissue to a first target depth by the actuator, and, controlling the hollow needle bodies to release electrical energy and perform inject in the first target depth based on the one or more selected modes. When completing the release of the electrical energy and completing injection, driving the hollow needle bodies to move from the first target depth to the second target depth by the actuator, and releasing electrical energy and performing inject in the second target depth, thereby meeting the requirements of different treatment depths in the dermis layer. At the same time, in this embodiment, it can further control the hollow needle bodies to release medium frequency electrical energy or low frequency electrical energy to relieve the user's pain.

Based on the above-mentioned methods for controlling the electrical treatment device in various embodiments, the third embodiment of the electrical treatment device of the present application is proposed.

In this embodiment, as shown in FIG. 5, the electrical treatment device includes a plurality of hollow needle bodies 1005, a needle seat 1004, an actuator 1003, a memory 1001, a processor 1002, and a program for controlling the electrical treatment device stored in the memory 1001 and executable on the processor; one ends of the plurality of the hollow needle bodies 1005 being provided on the needle seat 1004, and the actuator 1003 being connected to the needle seat 1004 and the processor 1002 respectively.

The processor 1002 is configured for driving opposite ends of the hollow needle bodies 1005 to penetrate at least one target depths of a skin tissue by the actuator.

The processor 1002 is further configured for controlling the hollow needle bodies to electrically connect to the electric energy output assembly to release the electric energy and/or to connect to the liquid storage unit to perform drug injection based on the one or more selected modes.

The present embodiment discloses that driving opposite ends of the hollow needle bodies to penetrate a skin tissue by an actuator, the hollow needle bodies being configured to release electrical energy and/or perform injection; determining one or more selected modes corresponding to the hollow needle bodies; and controlling the hollow needle bodies to release electrical energy and perform injection based on the one or more selected modes. The treatment process requires penetrating the skin tissue repeatedly for multiple times with the needle bodies, since the hollow needle bodies in this embodiment can be used for radiofrequency treatment and injection treatment both, thus simplifying the treatment process, reducing the times of penetrating the skin tissue with the needle bodies, and improving the treatment efficiency and the user's treatment experience.

In another embodiment, an electrical treatment device includes:
a plurality of needle bodies, a needle seat, an electric energy output assembly, a liquid storage unit, an actuator, a memory, a processor, and a program for controlling the electrical treatment device stored in the memory and executable on the processor; opposite ends of the plurality of needle bodies being provided on the needle seat; the processor is configured for:
driving ends of the needle bodies to penetrate at least one depths of the skin tissue by the actuator; and
controlling a plurality of the hollow needle bodies to connect to the liquid storage unit to perform injection and/or controlling a plurality of solid needle bodies to electrically connect to the electric energy output assembly for releasing electric energy.

In the present application, the terms “include”, “comprise” or any other variation thereof are intended to cover a non-exclusive inclusion, such that a process, method, article or system that includes a list of elements not only includes those elements, but also includes other elements not expressly listed, or also includes elements inherent to the process, method, article or system. In a case of without further restrictions, an element defined by the statement “includes . . . ” does not exclude the existence of other identical elements in a process, method, article or system that includes this element.

The serial numbers of the embodiments of the present application are only for description and do not represent the advantages and disadvantages of the embodiments. By the description of the above implementation mode, those skilled in the art can clearly understand that the above embodiment method can be implemented by means of software plus the necessary general hardware platform mode. Certainly, it can also be implemented by hardware, but in many cases the former is a better implementation mode. Based on this understanding, the technical solution of the present application, or the part that contributes to the prior art, can be embodied in the form of a software product, which is stored in a storage medium (such as a read-only memory/random access memory, a disk, or an optical disk), and includes several instructions for enabling a terminal device (which can be a mobile phone, a computer, a server, or a network device, etc.) to execute the methods of each embodiment of the present application. The above are only some embodiments of the present application, and do not limit the scope of the present application. Any equivalent structure or equivalent process transformation made using the contents of the specification and drawings of the present application, or directly or indirectly used in other related technical fields, are also included in the scope of the present application.

What is claimed is:

1. A method for controlling an electrical treatment device with an injection function, comprising:
driving ends of a plurality of needle bodies to penetrate at least one target depths of a skin tissue by an actuator; and
controlling the plurality of the needle bodies to electrically connect to an electric energy output assembly to release an electric energy at least one target depths of the skin tissue, and/or controlling the plurality of the needle bodies to connect to a liquid storage unit to perform drug injection based on at least one target depths of the skin tissue.

2. The method for controlling the electrical treatment device according to claim 1, wherein the plurality of the needle bodies comprise at least one or more hollow needle bodies and at least one or more solid needle bodies; the hollow needle bodies are capable of being connected to the electric energy output assembly and the liquid storage unit; the solid needle bodies are capable of being connected to the electric energy output assembly;
said controlling the plurality of the needle bodies to electrically connect to the electric energy output assembly to release the electric energy at least one target depths of the skin tissue; and/or controlling the plurality of the needle bodies to connect to the liquid storage unit to perform drug injection at least one target depths of the skin tissue further comprises:
determining one or more selected modes of the hollow needle bodies and the solid needle bodies; and
controlling the hollow needle bodies to release the electrical energy and perform injection, and controlling the solid needle bodies to release the electrical energy based on the one or more selected modes.

3. The method for controlling the electrical treatment device according to claim 2, wherein the one or more selected modes comprise a first work mode, the electrical energy comprises radiofrequency energy, and said controlling the hollow needle bodies to release the electrical energy and perform injection based on the one or more selected modes comprises:
in response to the one or more selected modes being selected in the first work mode, controlling the hollow needle bodies to release the electrical energy;
wherein the first work mode is that the hollow needle bodies release the electrical energy first and then perform injection.

4. The method for controlling the electrical treatment device according to claim 3, wherein the one or more selected modes further comprise a second work mode; said controlling the hollow needle bodies to release electrical energy and perform injection based on the one or more selected modes comprises:
in response to the one or more selected modes being selected in the second work mode, controlling the hollow needle bodies to perform injection;
wherein the second work mode is that the hollow needle bodies perform injection first and then release the electrical energy.

5. The method for controlling the electrical treatment device according to claim 2, wherein the hollow needle bodies and the solid needle bodies are disposed at intervals, and a length of each of the hollow needle bodies is different from a length of each of the solid needle bodies; said driving ends of the plurality of the needle bodies to penetrate at least one target depths of the skin tissue by the actuator comprises:
driving the ends of the hollow needle bodies and the ends of the solid needle bodies to penetrate the skin tissue by the actuator simultaneously.

6. The method for controlling the electrical treatment device according to claim 2, wherein the actuator is connected to the hollow needle bodies, the solid needle bodies and the processor respectively, and the processor is configured to control the hollow needle bodies and the solid needle bodies to move by the actuator; said driving ends of the plurality of the needle bodies to penetrate at least one target depths of the skin tissue by the actuator comprises:
driving the ends of the hollow needle bodies and the ends of the solid needle bodies to penetrate a first target depth of the skin tissue by the actuator;
said controlling the hollow needle bodies to release the electrical energy and perform injection, and controlling the solid needle bodies to release the electrical energy based on the one or more selected modes comprises:
controlling the hollow needle bodies to release the electrical energy and perform injection at the first target depth, and controlling the solid needle bodies to release the electrical energy based on the one or more selected modes;
in response to completing the releasing of the electrical energy and completing the injection, driving the hollow needle bodies to move from the first target depth to a second target depth of the skin tissue by the actuator;
wherein the first target depth is greater than the second target depth; and
controlling the hollow needle bodies to release the electrical energy and perform the injection at the second target depth, and controlling the solid needle bodies to release the electrical energy based on the one or more selected modes.

7. The method for controlling the electrical treatment device according to claim 6, wherein after said controlling the hollow needle bodies to release the electrical energy and perform the injection at the second target depth, and controlling the solid needle bodies to release the electrical energy based on the one or more selected modes, the method further comprises:
driving the solid needle bodies to continue to move to a third target depth of the skin tissue by the actuator;
wherein the third target depth is greater than the first target depth and the second target depth; and
controlling the solid needle bodies to release the electrical energy in modes with preset parameters during a movement; wherein the modes comprise a continuous release mode and a fractional release mode.

8. The method for controlling the electrical treatment device according to claim 7, wherein said driving ends of the plurality of the needle bodies to penetrate at least one target depths of the skin tissue by the actuator comprises:
driving the ends of the hollow needle bodies and the ends of the solid needle bodies to penetrate the first target depth of the skin tissue and/or the second target depth of the skin tissue by the actuator;
said controlling the hollow needle bodies to inject and controlling the solid needle bodies to release the electrical energy comprises:
controlling the hollow needle bodies to inject in the first target depth and/or the second target depth, and controlling the solid needle bodies to release the electrical energy;
driving the solid needle bodies to continue to move to the third target depth by the actuator; and
controlling the solid needle bodies to release the electrical energy in the preset parameter release mode during the movement.

9. The method for controlling the electrical treatment device according to claim 1, wherein the plurality of the needle bodies comprise a plurality of hollow needle bodies, ends of the plurality of the hollow needle bodies are provided on the needle seat, and the program for controlling the electrical treatment device is configured to implement the following steps:
driving the ends of the hollow needle bodies to penetrate at least one depths of the skin tissue by the actuator;
determining one or more selected modes corresponding to the hollow needle bodies; and
controlling the hollow needle bodies to connect to the electric energy output assembly to release the electric energy and/or to connect to the liquid storage unit to perform injection based on the one or more selected modes.

10. The method for controlling the electrical treatment device according to claim 9, wherein the actuator is connected to the needle seat and the processor respectively, and the processor is configured to drive the needle seat to move by the actuator; said driving the ends of the plurality of the hollow needle bodies to penetrate at least one target depths of the skin tissue by the actuator comprises:
driving the ends of the hollow needle bodies to penetrate a first target depth of the skin tissue by the actuator;
said controlling the hollow needle bodies to connect to the electric energy output assembly to release the electric energy and/or to connect to the liquid storage unit to perform injection based on the one or more selected modes comprises:
controlling the hollow needle bodies to release the electrical energy and/or perform injection in the first target depth based on the one or more selected modes;
driving the hollow needle bodies to move from the first target depth to a second target depth of the skin tissue by the actuator; wherein the first target depth is greater than the second target depth, and at least one the second target depths are provided; and
controlling the hollow needle bodies to release the electrical energy and/or perform injection in the second target depth based on the one or more selected modes.

11. The method for controlling the electrical treatment device according to claim 10, wherein said driving the ends of the hollow needle bodies to penetrate at least one depths of the skin tissue by the actuator comprises:
driving the hollow needle bodies to penetrate a third target depth of the skin tissue by the actuator, wherein the third target depth is greater than the first target depth and the second target depth;
said controlling the hollow needle bodies to release the electrical energy and/or perform injection based on the one or more selected modes comprises:
controlling the hollow needle bodies to release the electrical energy in the third target depth based on the one or more selected modes;
driving the hollow needle bodies to move from the third target depth to the first target depth and the second target depth respectively by the actuator; and
during the movement, controlling the hollow needle bodies to release the electrical energy in modes with preset parameters based on the one or more selected modes, and/or controlling the hollow needle bodies to perform at least one-shot injection at the first target depth, the second target depth, and during a process of moving from the first target depth to the second target depth; wherein the modes comprise a continuous release mode and a fractional release mode.

12. The method for controlling the electrical treatment device according to claim 11, wherein the method further comprises:
during the movement from the third target depth to the first target depth, controlling the hollow needle bodies to release the electrical energy and/or perform drug injection with the preset parameters, and controlling the hollow needle bodies to perform at least one-shot injection at the first target depth, the second target depth, and during the movement from the first target depth to the second target depth.

13. The method for controlling the electrical treatment device according to claim 1, further comprising:
driving the ends of the plurality of hollow needle bodies to penetrate at least one target depths of the skin tissue by the actuator; and
controlling the hollow needle bodies to release the electrical energy, or perform injection, or release the electrical energy and perform injection at the target depth based on the one or more selected modes.

14. The method for controlling the electrical treatment device according to claim 1, further comprising:
driving the ends of the plurality of hollow needle bodies to penetrate at least one target depths of the skin tissue by the actuator; and
controlling the hollow needle bodies to release the electrical energy firstly and then perform injection.

15. The method for controlling the electrical treatment device according to claim 1, further comprising:
driving the ends of the plurality of hollow needle bodies to penetrate at least one target depths of the skin tissue by the actuator; and
controlling the hollow needle bodies to perform injection.

16. The method for controlling the electrical treatment device according to claim 1, wherein the plurality of the needle bodies comprise at least one or more hollow needle bodies and at least one or more solid needle bodies; the hollow needle bodies are capable of being connected to the liquid storage unit, and the solid needle bodies are capable of being connected to the electric energy output assembly; and
said controlling the plurality of the needle bodies to electrically connect to the electric energy output assembly to release the electric energy at least one target depths of the skin tissue; and/or controlling the plurality of the needle bodies to connect to the liquid storage unit to perform drug injection at least one target depths of the skin tissue further comprises:
determining one or more selected modes of the hollow needle bodies and the solid needle bodies; and
controlling the hollow needle bodies to perform injection, and controlling the solid needle bodies to release the electrical energy based on the one or more selected modes.

17. The method for controlling the electrical treatment device according to claim 1, wherein the actuator comprises a vacuum pump inside the electrical treatment device connecting with a negative pressure pipe inside the tip, which is in fluid communication with a through hole opened on a front surface of the tip, for exerting attraction onto the treatment surface of the skin tissue towards the plurality of the needle bodies; said driving ends of the plurality of the needle bodies to penetrate at least one target depths of the skin tissue by the actuator comprises:
controlling the ends of the needle bodies to penetrate at least one depths of the skin tissue by the vacuum pump based on selected negative pressure modes.

18. The method for controlling the electrical treatment device according to claim 1, wherein the actuator comprises a first actuating motor configured for driving the needle bodies to move and a second actuating motor configured for injecting the drug from the liquid storage unit into the skin tissue; said driving ends of the plurality of the needle bodies to penetrate at least one target depths of the skin tissue by the actuator comprises:
driving the ends of the needle bodies to penetrate the skin tissue by the first actuating motor; and
said controlling the plurality of needle bodies to electrically connect to the electric energy output assembly to release the electric energy and/or to connect to the liquid storage unit to perform drug injection comprises:
controlling the needle bodies to release the electric energy based on the one or more selected modes, and/or controlling the needle bodies to inject by the second actuating motor.

19. An electrical treatment device, comprising:
a plurality of hollow needle bodies, a needle seat, an electric energy output assembly, a liquid storage unit, an actuator, a memory, a processor, and a program for controlling the electrical treatment device stored in the memory and executable on the processor; opposite ends of the plurality of the hollow needle bodies being provided on the needle seat, and the actuator being connected to the needle seat and the processor respectively; wherein the processor is further configured for:
driving ends of the hollow needle bodies to penetrate at least one target depths of a skin tissue by the actuator; and
controlling the hollow needle bodies to electrically connect to the electric energy output assembly to release the electric energy and/or to connect to the liquid storage unit to perform drug injection based on the one or more selected modes.

20. An electrical treatment device, comprising:
a plurality of needle bodies, a needle seat, an electric energy output assembly, a liquid storage unit, an actuator, a memory, a processor, and a program for controlling the electrical treatment device stored in the memory and executable on the processor; opposite ends of the plurality of needle bodies being provided on the needle seat; wherein the processor is configured for:
driving ends of the needle bodies to penetrate at least one depths of the skin tissue by the actuator; and
controlling a plurality of the hollow needle bodies to connect to the liquid storage unit to perform injection and/or controlling a plurality of solid needle bodies to electrically connect to the electric energy output assembly for releasing electric energy.
